(19) United States
(12) Patent Application Publication
(10) Pub. No.: US 2007/0281038 A1
(43) Pub. Date: Dec. 6, 2007

(54) Method of using a protein concentrate

(76) Inventor: Andre Trouille, Bienenbuttel (DE)

Correspondence Address: WILLIAM COLLARD;COLLARD & ROE, P.C., 1077 NORTHERN BOULEVARD, ROSLYN, NY, 11576, US

(21) Appl. No.: 11/809,231

(22) Filed: May 31, 2007

(30) Foreign Application Priority Data
May 31, 2006 (DE) 10 2006 025 338.8

(51) Int. Cl. A61K 36/48; A61K 35/20

(52) U.S. Cl. 424/535; 424/757

(57) ABSTRACT

A method for treating metabolic disorders or illnesses associated with metabolic disorders by administering a protein-rich nutrient concentrate as a dietetic food. Studies show that the administration of concentrate leads to highly significant normalization of numerous parameters that are considered to be risk factors or markers for cardiovascular illnesses and atherosclerosis.

### BACKGROUND OF THE INVENTION

1. Field of the Invention

The invention relates to a method of treating metabolic disorders by administering an easily digestible protein concentrate.

2. The Prior Art

In patients having underlying illnesses such as diabetes mellitus, for example, disorders of the metabolism are also frequently present. Thus, diabetes as an illness is characterized by disorders of the lipid, carbohydrate, and insulin metabolism. In order to have an advantageous influence on these metabolic processes with regard to the progression of the illness, adherence to a diet is necessary. In the case of less serious illness, the diet alone can be sufficient, or it can support treatment with medications. In this connection, within the framework of the diet, the patient refrains from eating animal products, such as fish, meat, sausages, eggs, to a great extent, since these foods have a high proportion of cholesterol and triglycerides. It is a disadvantage of this diet that the lower consumption of proteins can result in a deficiency of essential amino acids. These amino acids cannot be synthesized by the body itself. Specifically in the case of metabolic disorders, however, the essential amino acids must be consumed in a sufficient amount and in a correct ratio.

### SUMMARY OF THE INVENTION

It is therefore an object of the present invention to provide a nutrient that avoids the disadvantages of a conventional diet and is able, as a dietetic nutrient, to improve the metabolic situation of patients suffering from metabolic disorders.

This task is accomplished, according to the invention, by administering a protein-rich nutrient concentrate, the production of which is described in European Patent No. EP 1 049 384 B1. The concentrate has a protein proportion of at least 50 wt %. The protein is preferably present by at least 40% in the form of free amino acids or short-chain peptides.

Because of the high proportion of free amino acids and short-chain peptides, the nutrient concentrate is particularly easily digestible, because the proteins are already present as ingredients that are well absorbed in the intestine. This increases the tolerance, and this is particularly important for persons who are ill.

The protein concentrate is produced from a vegetable starting product, preferably soy or a soy product, particularly having a protein proportion of 60 to 95%, or an animal starting product, preferably milk or a milk product, or a mixture of the aforementioned vegetable and animal starting products. The starting product or the mixture is impregnated with an emulsifier, preferably lecithin, in an amount of 1 to 6 wt %, preferably 2 to 4 wt %, and then honey is stirred into protein-rich protein concentrate impregnated in this manner, in a proportion of 10 to 40 wt %, preferably 25 to 30 wt %. The honey is preferably liquid or present as an aqueous solution, and is added in a very fine jet, preferably from several nozzles and particularly without pressure. A temperature of 60° C., and preferably room temperature, is not exceeded, and the protein concentrate is allowed to stand for several hours after the honey has been stirred in, preferably until a moisture content of 1 to 8 wt %, particularly 2 to 5 wt %, has been reached.

In this manner, a yellowish powder having a slightly nutty taste is obtained, which has a moisture content of approximately 7% by weight. The yield is practically quantitative. The heating observed during mixing does not result in any destruction, particularly of the essential amino acids. The powder has the following amino acid content:

| Amino acid | g/100 g |
|---|---|
| Alanine | 1.9 |
| Arginine | 3.8 |
| Asparaginic acid | 6.1 |
| Cysteine | 3.5 |
| Glutaminic acid | 10.6 |
| Glycine | 2.9 |
| Histidine | 1.1 |
| Isoleucine | 1.2 |
| Leucine | 4.7 |
| Lysine | 3.1 |
| Methionine | 2.1 |
| Phenylalanine | 3.1 |
| Proline | 2.6 |
| Serine | 5.7 |
| Threonine | 2.3 |
| Tryptophan | 0.30 |
| Tyrosine | 2.7 |
| Valine | 2.4 |

It has been shown that the protein concentrate produced according to the method described above is suitable for treating patients suffering from metabolic disorders or from illnesses that are associated with metabolic disorders. These can be, for example, disorders of the amino acid metabolism. Because of the high proportion of free amino acids, the concentrate counteracts deficiency symptoms, in that sufficient essential and non-essential amino acids are made available. However, the influence of the concentrate is not limited to this effect, since the different physiological metabolic processes are regulative and are coupled by way of substrate exchange. Thus, amino acids can be precursors for other metabolic products. Asparaginic acid, for example, can stimulate the synthesis of ATP, in that it is converted to oxalacetate and then passed into the citrate cycle. In addition to this direct effect of the ingredients, the protein concentrate also influences other disordered physiological processes, such as the lipid metabolism and carbohydrate metabolism, by means of its dietetic effect.

### BRIEF DESCRIPTION OF THE DRAWINGS

Other objects and features of the present invention will become apparent from the following detailed description considered in connection with the accompanying drawing. It is to be understood, however, that the drawing is designed as an illustration only and not as a definition of the limits of the invention.

FIG. 1 shows a bar chart that reproduces the Body Mass Index (BMI) in kg/m² and the body fat proportion in percent, before and after treatment with the method according to the invention.

### DETAILED DESCRIPTION OF THE EMBODIMENTS

The invention will be described with reference to the following examples:

It is possible to treat diabetes mellitus, preferably type 2, using the nutrient concentrate. The effect is due to the fact that the metabolic situation of diabetics is improved, and, in particular, the glucose level is lowered.

This was shown by the following study:

Thirty women, mean age 63.3 years, and 30 men, mean age 63.8 years, all of whom were not insulin-dependent, but were being treated with oral medication and/or a diet plan, consumed 50 grams of the concentrate daily, without any other change in medication or lifestyle. At the beginning of the study, usual laboratory blood parameters, such as fasting glucose, insulin, HbAlC, and body weight of the participants were measured. The glucose tolerance was determined after 12 hours of fasting, with a drink of 75 grams glucose in 150 ml water, after one and two hours. The measurement of fasting glucose and insulin was repeated after eight and 20 weeks, and the measurement of the parameters fasting glucose, HbAlC, insulin, weight, and glucose tolerance were repeated after 14 and 26 weeks, in each instance. Within 26 weeks, the fasting glucose level dropped from an average of 167 mg/dl to 127 mg/dl, the HbAlC from 8.2 to 7.3 percent, and the insulin level, measured after fasting, from 20.7 mU/ml to 10.6 mU/ml. The weight of the diabetics dropped by 2.5 to 3.7 kg during the study period of 26 weeks.

Furthermore, the protein concentrate can be used for the treatment of patients suffering from hyperlipidemia. This was shown by the following study:

In the case of twelve overweight persons (seven men, five women, age 34-66 years, BMI>25 kg/m²), the following characteristic values were determined within the framework of a therapeutically indicated weight intervention, at the beginning and after four weeks:

Anthropometric data: height, weight, body fat, BMI.

Lipid status: cholesterol; TG, HDL, LDL, VLDL, Apo Al, Apo All, Apo B, Lp (a).

Concomitant proinflammatory factors: leptin, insulin, CRP, fibrinogen.

Concomitant metabolic regulation: TSH, STH.

Renal or hepatic functional disorder or a hormonal cause of the overweight were precluded. During an intervention phase that lasted for four weeks, at first, the participants received an individual dosage of the protein concentrate according to the invention, coordinated in accordance with their normal weight. Aside from the consumption of the dietary nutrient in the morning and evening, the consumption of a fat-reduced normal meal was permitted at midday. With this, the daily calorie consumption was approximately 1200 kcal/day.

The parameters of lipid metabolism, which were measured at the beginning and after four weeks of administration of the protein concentrate, are shown in Table 1; significant differences are characterized with the symbol * and **, corresponding to p<0.05 and <0.01, respectively:

TABLE 1

| | Beginning | 4 weeks |
|---|---|---|
| Total cholesterol (mg/dl) | 199.0 ± 28.7 | 161.0 ± 26.7** |
| Triglycerides (mg/dl) | 160.0 ± 53.4 | 125.0 ± 63.4* |
| HDL cholesterol (mg/dl) | 48.4 ± 11.4 | 45.0 ± 7.5 |
| LDL cholesterol (mg/dl) | 112.7 ± 25.4 | 87.6 ± 22.9* |
| VLDL cholesterol (mg/dl) | 33.7 ± 10.6 | 23.5 ± 7.42* |
| Apo Al (mg/dl) | 160.0 ± 20.2 | 148.0 ± 24.1 |
| Apo All (mg/dl) | 47.0 ± 5.3 | 38.4 ± 5.9** |
| Apo B (mg/dl) | 109.4 ± 25.9 | 84.6 ± 18.3** |
| Lp-a cholesterol (mg/dl) | 4.0 ± 7.3 | 5.3 ± 9.5 |

### Hormones of weight regulation and concomitant proinflammatory and metabolic/hormonal parameters are listed in Table 2 (symbols as in Table 1):

TABLE 2

| | Beginning | 4 weeks |
|---|---|---|
| Leptin (ng/ml) | 24.60 ± 15.80 | 13.80 ± 13.20** |
| Insulin (mU/ml) | 14.20 ± 9.30 | 7.30 ± 9.00* |
| CRP (mg/dl) | 0.24 ± 0.20 | 0.13 ± 0.12* |
| Fibrinogen (mg/dl) | 371.00 ± 49.40 | 366.00 ± 61.40 |
| TSH (mg/ml) | 0.90 ± 0.87 | 0.72 ± 0.74 |
| STH (ng/ml) | 0.89 ± 1.48 | 0.65 ± 0.74 |

During the intervention and the related weight reduction, the leptin level clearly drops. In this connection, the leptin level is lowered more with the weight decrease achieved with the administration of the concentrate than with the weight decrease achieved with a conventional diet. Since increased leptin levels are considered to be causes for adiposity and metabolic disorders, such as diabetes, a central risk factor can thereby be positively influenced.

As Table 2 furthermore shows, the insulin concentration in the blood is significantly reduced under the influence of the protein concentrate. Elevated insulin values are considered to be an atherogenic and inflammatory risk factor.

Furthermore, a highly significant reduction in the total cholesterol and the LDL cholesterol occurs. In addition to the atherogenic lipid profile, changes in the proinflammatory profile are also observed. The lipoprotein Apo-B, which is particularly atherogenic for the blood vessel walls, is reduced more in the groups that received concentrate than in the comparison groups, by a significant amount.

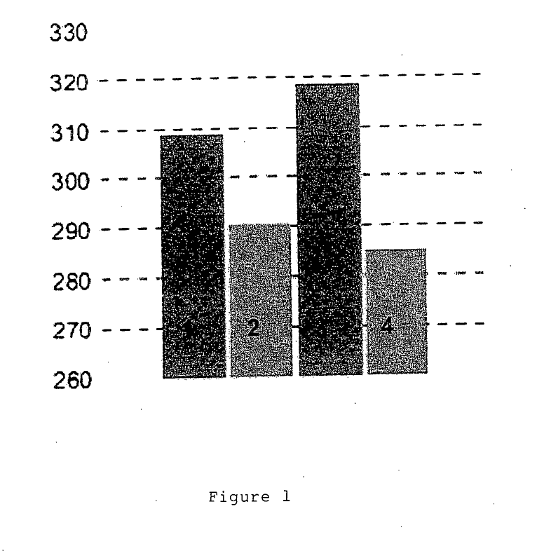

FIG. 1 shows a bar chart that reproduces the Body Mass Index (BMI) in kg/m² and the body fat proportion in percent, before and after the intervention, in other words the consumption of the protein concentrate, in each instance. Bar 1 shows the BMI and Bar 3 shows the body fat proportion before the intervention, Bar 2 shows the BMI and Bar 4 shows the body fat proportion after the intervention. The figure discloses that in the case of all the persons studied, a significant weight reduction was documented after four weeks. The mean weight loss was 4.8 kg.

These results can be summarized to state that the protein-rich nutrient concentrate has surprisingly advantageous effects on the metabolic situation of patients suffering from diabetes mellitus or hyperlipidemia.

The administration of the concentrate results in highly significant normalization of numerous parameters that are considered to be risk factors or markers for cardiovascular illnesses and atherosclerosis.

Accordingly, while only a few embodiments of the present invention have been shown and described, it is obvious that many changes and modifications may be made thereunto without departing from the spirit and scope of the invention.

What is claimed is:

1. A method for treating metabolic disorders in patients by administering to the patients a protein-rich nutrient concentrate having a protein content of at least 50 wt %, wherein the concentrate is produced according to the following method:
providing a vegetable starting product, an animal starting product, or a mixture of vegetable and animal starting products;
impregnating the starting product with an emulsifier in an amount of 1 to 6% wt %; and
stirring honey into the impregnated starting product, in a proportion of 10 to 40 wt %,
wherein a temperature of 60° C. is not exceeded, and the protein concentrate is allowed to stand for several hours after the honey has been stirred in.

2. A method to claim 1, wherein the disorder is diabetes mellitus.

3. A method according to claim 1, wherein the disorder is hyperlipidemia.

4. The method according to claim 1, wherein the emulsifier is lecithin.

5. The method according to claim 1, wherein the starting product is a soy product.

6. The method according to claim 1, wherein the starting product is milk or a milk product.

7. The method according to claim 1, wherein the honey is liquid or aqueous solution and is added in a fine jet to the emulsified starting product from several nozzles and without pressure.

8. The method according to claim 1, wherein the concentrate is allowed to stand until a moisture content of 1 to 8% by weight has been reached.

9. The method according to claim 1, wherein the concentrate has protein in the form of free amino acids or short-chain peptides in an amount of at least 40 wt %.
